(19) United States
(12) Patent Application Publication
(10) Pub. No.: US 2012/0040449 A1
(43) Pub. Date: Feb. 16, 2012

(54) DISPOSABLE BIOREACTOR AND USE-ONCE STIRRING SYSTEM

(76) Inventor: Jean Pascal Zambaux, Aubenge (FR)

(21) Appl. No.: 13/259,576

(22) PCT Filed: Mar. 24, 2010
(86) PCT No.: PCT/FR10/50534

§ 371 Date: Oct. 26, 2011

(30) Foreign Application Priority Data
Mar. 24, 2009 (FR) 0951895

(51) Int. Cl. C12M 1/02; C12M 1/06

(52) U.S. Cl. 435/302.1; 435/283.1

(57) ABSTRACT

The present invention relates to a disposable bioreactor including a flexible pouch made of a polymer material and in addition a single-use stirring system placed entirely within said pouch; said stirring system comprising a drive means and at least one stirring means driven in rotation by said drive means via magnetic coupling. The invention also relates to a single-use stirring system.

The present invention pertains to a disposable bioreactor, as well as to a single-use stirring system.

### STATE OF THE ART

A bioreactor includes reactors employed in fermentation, in enzymatic reaction, in cell culture or used in the field of tissue engineering, in the manufacture of biological or chemical products, of biological medicines and of microorganisms. The manufacture of these products requires steps consisting of cleaning, of sterilization and of validation of the equipment such as bioreactors and stirring systems, extremely rigorous and demanding steps which substantially increase the cost of manufacturing these products. Sterile and disposable bioreactors and stirring systems have consequently been developed to correct this problem, particularly with the aim of minimizing, cleaning and validation times and/or avoiding the risk of contamination.

However, single-use stirring systems currently available on the market have the disadvantage of being suited only to one given type of disposable bioreactor. The user of a given disposable bioreactor is therefore compelled to acquire a single-use stirring system from the manufacturer selling said given disposable bioreactor, often at a relatively high price.

Thus, there exists a real need to offer a disposable bioreactor which offers an alternative solution to existing bioreactors, as well as a single-use stirring system which does not have the aforementioned disadvantage.

### OBJECTS OF THE INVENTION

The invention has as its principal object to offer a disposable bioreactor with a simple design, as well as a single-use stirring system which solves the aforementioned problem, that is one which is suited to any disposable bioreactor.

The disposable bioreactor as well as the single-use stirring system according to the present invention must have the advantage of being usable on an industrial and commercial scale, safely and reliably, and their simple structure must allow the manufacture of a disposable bioreactor and of a cheap single-use stirring system, both at low cost.

### SUMMARY OF THE INVENTION

In accordance with a first object, the invention relates to a disposable bioreactor including a flexible pouch made of a polymer material. The bioreactor also includes a single-use stirring system placed entirely within said pouch; said stirring system comprising a drive means and at least one stirring means driven in rotation by said drive means through magnetic coupling.

In other words, said drive means and said at least one stirring means of the stirring system are placed entirely within the flexible pouch made of polymer material. The polymer material is advantageously selected from among the group consisting of polyethylene, polypropylene, the polyamides, polyether etherketone (PEEK), the copolymer of ethylene and polyvinyl alcohol (EVOH) and any plastic including a bilayer surface coating, said bilayer including a primary internal layer of hydroxypropylmethyl cellulose (HPMC) or polyvinyl alcohol (PVA) and a biologically active external layer located on top of said internal layer. The bilayer coating constitutes a barrier between the plastic constituting the pouch and the medium to be mixed in the pouch (advantageously a cell culture medium) and prevents the attachment of proteins (advantageously cells) to the plastic. The nature of this bilayer coating, as well as its use, are more particularly described in patent FR 2 862 979 B1.

Generally, the term “disposable” is understood to mean that the bioreactor is sterile before use and that it is discarded after use. The expression “single use” must be understood to indicate that the stirring system is sterile before its use and that it can be used several times before being thrown away, as long as the reactions in which it is used to mix the medium take place under identical conditions (same reactants, same quantity of reactants, same solvent . . . ).

According to one advantageous embodiment of the invention, the stirring system also includes a means for actuating the drive means consisting of a fluid or electricity (electrical charge) supply line.

According to one particularly advantageous embodiment of the invention, the fluid is a gas selected from the group consisting of air, nitrogen, carbon dioxide, argon, diatomic oxygen and any combination of these. The use of carbon dioxide, of diatomic oxygen, of nitrogen or of air as the fluid is advantageously preferred. Indeed, each of these gases can also be used to aerate the medium to be mixed in the bioreactor using an aerator and hence serve as a nutrition component for growing microorganisms.

According to one particularly advantageous embodiment of the invention, the disposable bioreactor also includes at least one tube running through the wall of the pouch. Advantageously, said at least one tube is attached or connected to the stirring system at the drive means. In a particularly preferred fashion, the at least one tube constitutes said means of actuating the drive means.

According to an advantageous embodiment of the invention, said at least one stirring means includes a rotation axle, said rotation axle having at least one metal surface capable of being attracted by a magnetized surface, preferably a magnet, and/or said rotation axle has at least one magnetized surface, preferably at least one magnet. Advantageously, the rotation axle includes either a metal surface or a magnetized surface on substantially its entire surface, or metal surfaces alternating with magnetized surfaces.

According to another advantageous embodiment of the invention, said at least one stirring means includes at least one propeller having a hub and at least one blade, and preferably two propellers. Advantageously, said at least one propeller includes several blades of different possible shapes. In the case where said at least one stirring means has several propellers, each propeller can include a different number of blades, possibly of different shapes.

According to one other advantageous embodiment of the invention, said drive means is a turbine which includes vanes integral with a hollow shaft, said rotation axle of said at least one stirring means being placed within the hollow of the shaft and the inner wall of said hollow shaft having:
at least one metal surface attracted by said magnetized surface in the rotation axle of said at least one stirring means; and/or
at least one magnetized surface, preferably at least one magnet, attracting said at least one metal or magnetized surface of the rotation axle of said at least one stirring means.

Thus, the inner wall of the hollow shaft is advantageously provided with either one or several magnetized surface(s) cooperating with one or several metal or magnetized surface(s) of the rotation axle. In a particularly advantageous manner, when the rotation axle includes a metal surface over substantially its entire surface, the inner wall of the hollow shaft has at least one magnetized surface cooperating with this metal surface of the rotation axle; or when the rotation axle includes an magnetized surface of polarity A over substantially its entire surface, the inner wall of the hollow shaft has at least one metal surface or at least one magnetized surface of opposite polarity to cooperate with the magnetized surface of the rotation axle so as to transmit rotational motion when the drive means is set in rotation.

Preferably, said at least one metal surface of said at least one magnetized surface of the inner wall of the hollow shaft is positioned on or in the inner wall of said hollow shaft, in line with the position of the vanes.

According to one particularly advantageous embodiment of the invention, the turbine also includes a fixed body having substantially the shape of a closed cylinder, said fixed body forming an enclosure wherein the vanes are able to move. By the expression “having substantially the shape of a closed cylinder” is meant a closed cylinder perforated at the center of the disks constituting it, either to its full height or partially, that is to say that one of these two disks is not perforated. The space defined by this perforation is advantageously of cylindrical shape and allows the rotation axle of the stirring means to be housed there.

According to another particularly advantageous embodiment of the invention, at least one of said vanes is provided with at least one magnet cooperating with at least one counter-magnet positioned on the lower surface of said at least one stirring means to prevent friction between the drive means and said at least one stirring means, said lower surface corresponding to the surface of said at least one stirring means opposite the surface from which said at least one vane of said at least one stirring means projects. Said at least one magnet is, by preference, positioned on one or more vane(s) in an inner peripheral region, that is near the rotation axis of the hollow shaft, but is not situated on or in the wall of the hollow shaft.

According to one advantageous variation in implementation of the invention, the fixed body of the turbine, in the shape of a cylinder, is provided with one or more magnet(s) in the outer peripheral region of the surface of the disk(s) of said cylinder. In a particularly advantageous Manner; this(these) magnet(s) has(have) a polarity identical to said at least one counter-magnet of the stirring means in order to prevent friction between the drive means and said at least one stirring means. Preferably, this(these) magnet(s) also has(have) a polarity identical to the magnet or magnets positioned on the vanes to more effectively repel the lower surface of said at least one stirring means.

According to yet another particularly advantageous embodiment of the invention, said fluid supply line also serves as an aerator for the medium to be mixed within the pouch. This embodiment is particularly preferred when the actuation means of the drive means is a gas. The presence of the aerator is even more advantageous when the selected gas contributes to the growth of microorganisms.

According to one advantageous embodiment of the invention, the height of said single use stirring system in the pouch is variable and can be varied during stirring. The mixing of the reaction medium can consequently be carried out homogeneously and can allow efficiency to be optimized.

According to one advantageous variation in implementation of the invention, the height of the single-use stirring system in the pouch can be modified thanks to the presence of attachment means of the stirring system when the stirring system is in use or when it is not operating.

The single use stirring system can therefore be held stably in the flexible pouch of the disposable bioreactor by means of an attachment means.

According to one particularly advantageous variation in implementation of the invention, the attachment means allows fluid or electricity supply lines to be connected at the top of the pouch. In this case, the attachment means consists of a male part and of a female part connected by a metal joint. The female part made of plastic is welded to the pouch. The plastic material can advantageously be selected from among the group consisting of polyethylene, polypropylene, the polyamides, polyether etherketone (PEEK), the copolymer of ethylene and polyvinyl alcohol (EVOH). The male part is provided with two holes wherein the fluid or electricity supply tubes are set in a substantially fixed fashion (the height of the tubes can however be modified under the influence of a physical force), and is placed over the female part. The metal connecting joint, consisting of two semicircles capable of pivoting on a common axis and a screw closure, allows the female and male parts to be joined into a single block, the block being combined with the flexible pouch. The fluid or electricity supply tubes can sometimes be placed within reinforcing bars to make them stiffer and stronger. The fluid or electricity supply tubes are advantageously held to a fixed base by means of a clamp-nut system. In particularly preferred fashion, the tubes are fluid supply tubes.

According to another particularly advantageous variation in implementation of the invention, the attachment means allows the fluid or electricity supply tubes to be connected at one side of the pouch. The attachment means consists of a dual connector for tubes. This connector takes the form of two ganged sprinkler hose connectors, that is to say that it consists essentially of two male parts of tubular shape each having a threaded end. These male parts also include, perpendicular to their tube, a substantially flat surface made of plastic allowing the dual connector to be welded to the pouch. The plastic material of the substantially flat surface can advantageously be selected from among the group consisting of polyethylene, polypropylene, the polyamides, polyether etherketone (PEEK), the copolymer of ethylene and polyvinyl alcohol (EVOH). The threaded shape of the ends allows a fluid-tight connection to the fluid or electricity supply tubes. In a particularly preferred fashion, the tubes are fluid supply tubes. Reinforcing bars can be connected to the non-threaded ends and be held to the fixed base by means of a clamp-nut system.

Thanks to these various attachment means, the stirring system can be attached in a non-permanent way within a pouch and/or withdrawn from a pouch after use to be attached within another pouch. In other words, the stirring system is adapted to and/or can match any disposable bioreactor through these attachment means.

According to its second object, the invention concerns a single-use stirring system comprising:
a drive means, and
at least one stirring means driven in rotation by said drive means via magnetic coupling,
   said at least one stirring means including a rotation axle. Said rotation axle has at least one metal surface capable of being attracted by a magnetized surface, preferably a magnet, and/or said rotation axle has at least one magnetized surface, preferably at least one magnet. Said drive means is a turbine which includes vanes that are an integral part of a hollow shaft. The rotation axle of said at least one stirring means is placed within said hollow portion of the shaft and the inner wall of said hollow shaft has:
at least one metal surface attracted by said magnetized surface of the rotation axle of said at least one stirring means; and/or
at least one magnetized surface, preferably at least one magnet, attracting said at least one metal or magnetized surface of the rotation axle of said at least one stirring means.

Advantageously, the rotation axle includes either a metal surface or a magnetized surface over substantially its entire surface, or metal surfaces alternating with magnetized surfaces.

The inner wall of the hollow shaft is provided either with one or with several magnetized surface(s) cooperating with one or several metal or magnetized surface(s) of the rotation axle, or with one or with several metal surface(s) cooperating with one or several magnetized surface(s) of the rotation axle. In a particularly advantageous manner, when the rotation axle includes a metal surface over substantially its entire surface, the inner wall of the hollow shaft has at least one magnetized surface cooperating with this metal surface of the rotation axle; or when the rotation axle includes a magnetized surface with polarity A over substantially its entire surface, the inner wall of the hollow shaft has at least one metal surface or at least one magnetized surface of polarity opposite to pole A to cooperate with the magnetized surface of the rotation axle so as to transmit rotational motion when the drive means is set in motion.

Preferably, said at least one metal surface or said at least one magnetized surface of the inner wall of the hollow shaft is positioned on or in the wall of said hollow shaft, in line with the location of the vanes.

According to one advantageous embodiment of the invention, said at least one stirring means includes at least one propeller having a hub and at least one blade, and preferably two propellers. Advantageously, said at least one propeller includes several blades of various possible shapes. In the case where said at least one stirring means has several propellers, each of the propellers can include a different number of blades, possibly of different shapes.

According to an advantageous embodiment of the invention, at least one of said vanes is provided with at least one magnet cooperating with at least one counter-magnet positioned on the lower surface of said at least one stirring means to prevent friction between the drive means and said at least one stirring means, said lower surface corresponding to the surface of said at least one stirring means opposite the surface from which said at least one vane of said at least one stirring means projects. Preferably, said at least one magnet placed on one or more vane(s) is preferably positioned in an inner peripheral, region of the vanes, that is near the axis of rotation of the hollow shaft, but is not situated on or in the wall of the hollow shaft.

According to another advantageous embodiment of the invention, the turbine also includes a fixed body having substantially the shape of a closed cylinder, said fixed body forming an enclosure wherein the vanes are free to move.

According to one advantageous variation of implementation of the invention, the fixed body of the turbine, in the shape of a cylinder, is provided with one or several magnet(s) in the outer peripheral region of the disk(s) of said cylinder. In a particularly advantageous manner, this or these magnets has (have) a polarity identical to the at least one counter-magnet of the stirring means in order to prevent friction between the drive means and the at least one stirring means. Preferably, this or these magnets also has (have) a polarity identical to the magnet or magnets positioned on the vanes to more effectively repel the lower surface of said at least one stirring means.

According to another advantageous embodiment of the invention, the stirring system also includes a means of actuating the drive means consisting of a fluid or electricity (electrical charges) supply line.

According to an advantageous variation of implementation of the invention, the fluid is a gas selected from among the group consisting of air, nitrogen, carbon dioxide, argon, diatomic oxygen and any mixture of these.

According to an advantageous embodiment of the invention, said fluid supply line also serves as an aerator for the medium to be mixed in the pouch. The use of carbon dioxide, of diatomic oxygen, of nitrogen, of air or of a mixture of these gases as an aerating fluid is advantageously preferred because each of these gases can serve as a nutritional component for growing microorganisms.

According to another advantageous embodiment of the invention, the height of said single-use stirring system within the pouch is variable and can be varied during stirring.

The invention as previously defined and as resulting from the description of the following figures showing currently preferred embodiments of the invention makes it possible to resolve the technical problems stated in the objects of the invention and thus allows the manufacture of a low-cost bioreactor and single-use stirring system of simple design, usable on an industrial and commercial scale.

Other aims, features and advantages of the invention will appear clearly in the light of the explanatory description which follows, made with reference to the currently preferred embodiments of the invention, forming an integral part of the invention but given only by way of illustration and which may not limit in any way the scope of the invention.

### DESCRIPTION OF FIGURES

FIG. 1 shows a perspective view of the single-use stirring system according to the invention and according to a currently preferred embodiment.

FIG. 2 shows a longitudinal section view of the single-use stirring system according to the present invention and according to a currently preferred embodiment.

FIG. 3 shows a perspective view of the disposable bioreactor according to the present invention and according to a first currently preferred embodiment.

FIG. 4 shows a longitudinal section view of a means of attaching the single-use stirring system within the flexible pouch of the disposable bioreactor according to the present invention and according to the first currently preferred embodiment.

FIG. 5 shows a perspective view of the disposable bioreactor according to the present invention and according to a second currently preferred embodiment.

FIG. 6 shows a longitudinal section view of a means of attaching the single-use stirring system within the flexible pouch of the disposable bioreactor according to the present invention and according to the second currently preferred embodiment.

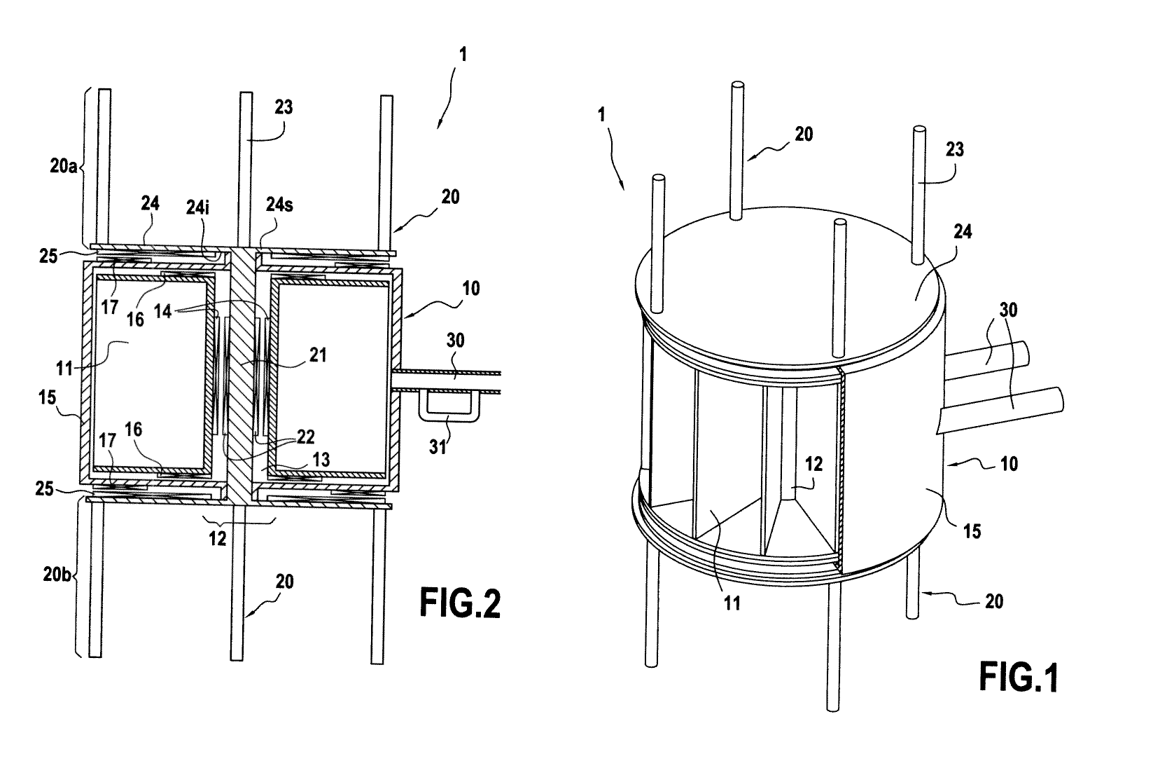

Referring to FIGS. 1 and 2, a currently preferred embodiment of a single-use stirring system according to the present invention is shown.

The single-use stirring system 1 includes:
a drive means 10, and
a stirring means 20 driven in rotation by said drive means 10 via magnetic coupling,
   said stirring means 20 including a rotation axle 21. The rotation axle 21 has at least two magnets 22. The drive means is a turbine 10 which includes vanes 11 that are an integral part of a hollow shaft 12. The rotation axle 21 of the stirring means 20 is placed in the hollow 13 of the shaft 12 and the wall of said hollow shaft 12 has, in line with each vane 11, a magnet 14 which attracts one of said at least two magnets 22 of the rotation axle 21 of the stirring means 20. The magnets 14 placed on the wall of said hollow shaft 12 and the magnets 22 of the rotation axle therefore have opposite polarity in order to attract one another.

As illustrated in. FIGS. 1 and 2, the turbine 10 also includes a fixed body 15 having substantially the shape of a closed cylinder 15, said fixed body 15 forming an enclosure wherein the vanes 11 are free to move.

FIGS. 1 and 2 show that the stirring means 20 includes two propellers 20a, 20b having a hub 24 and four blades 23. The first propeller can of course have a different number of blades, possibly of different shapes, compared with the second propeller.

FIGS. 1 and 2 also show that each vane 11 is provided with a magnet 16 cooperating with a counter-magnet 25 positioned on the lower surface 24i of the stirring means 20 to prevent friction between the drive means 10 and the stirring means 20, said lower surface 24i corresponding to the surface of said at least one stirring means opposite the surface 24s from which the blades 23 of the stirring means extend. The magnet 16 set on the vane 11 is positioned in an inner peripheral region of the vanes, that is near the axis of rotation of the hollow shaft 12, but is not situated on or en the wall of the hollow shaft.

As illustrated in FIGS. 1 and 2, the fixed body 15 of the turbine 10, in the shape of a cylinder, is provided with several magnets 17 in the outer peripheral region of the surface of each of the disks of said cylinder. These magnets 17 have a polarity identical to that of the counter-magnets 25 of the stirring means 20 in order to prevent friction between the drive means 10 and said at least one stirring means 20. In addition, these magnets 17 also have a polarity identical to the magnets 16 positioned on the vanes 11 to more effectively repel the lower surface 24i of said at least one stirring means 20.

FIGS. 1 through 6 show that the stirring system 20 also includes a means 30 for actuating the drive means 10 consisting of a fluid supply line. The fluid is a gas which can be selected from among the group consisting of air, nitrogen, carbon dioxide, argon, diatomic oxygen and any mixture of these. The gas supply line also serves as an aerator 31 for the medium to be mixed. This latter configuration can allow the gas that is used to constitute a contribution of nutritional components to growing microorganisms.

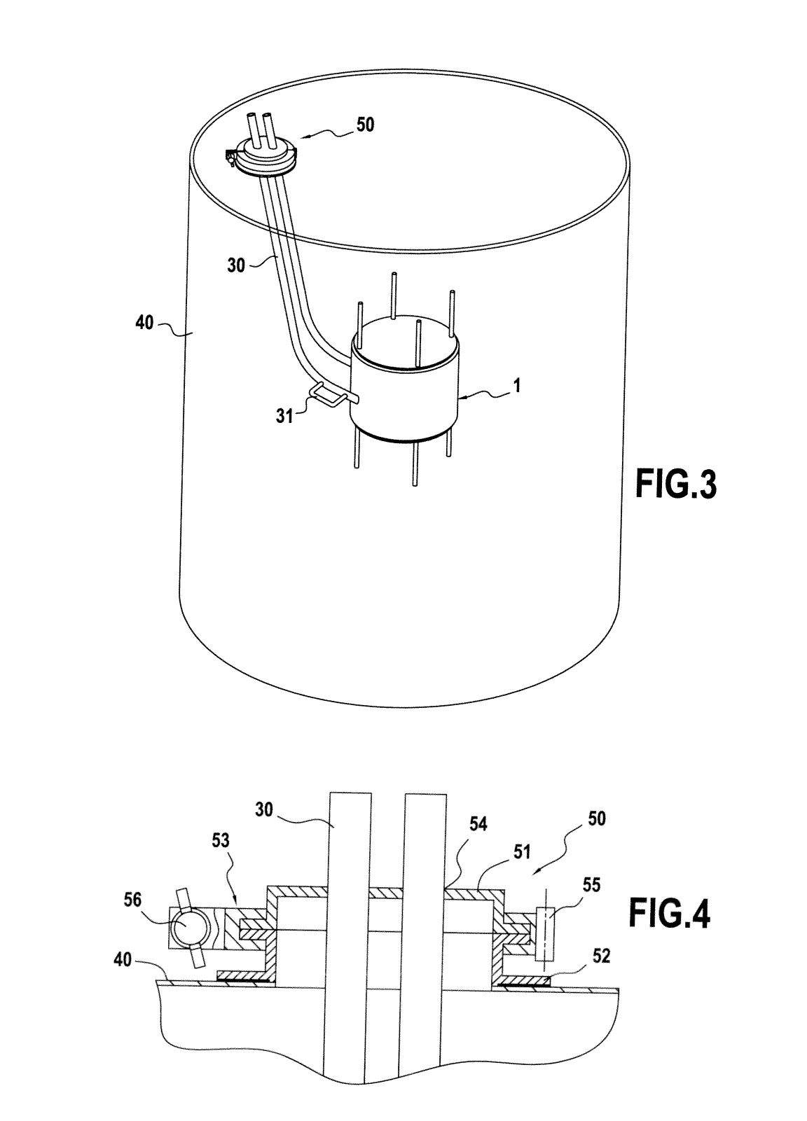

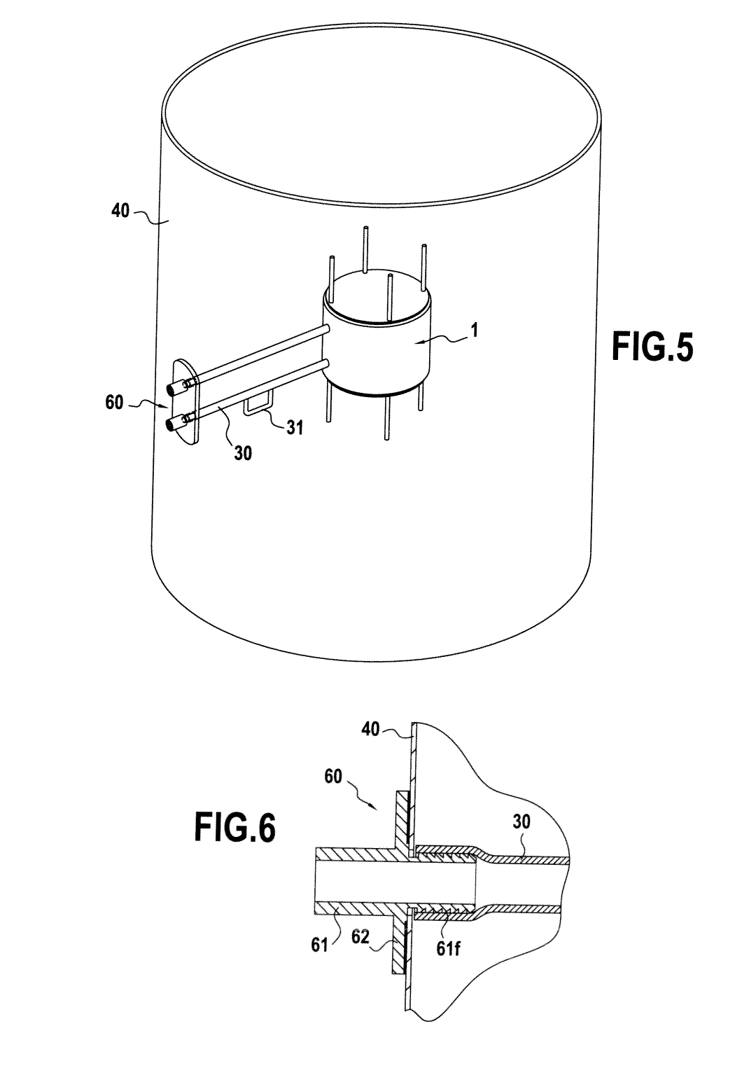

Referring to FIGS. 3 through 6, particularly to FIGS. 3 and 5, first and second currently preferred embodiments of a disposable bioreactor according to the present invention are shown.

The disposable bioreactor of each of these embodiments includes a flexible pouch 40 made of a polymer material and a single-use stirring system 1 as previously described.

As shown in FIGS. 3 through 6, the single-use stirring system 1 is held stably to the flexible pouch 40 of the disposable bioreactor by means of an attachment means 50; 60. This attachment means 50; 60 makes it possible to modify the height of the single-use stirring system 1 in the pouch 40 when the single-use stirring system 1 is in use or when it is not operating.

FIGS. 3 and 4 illustrate the attachment means 50 according to the first currently preferred embodiment of the disposable bioreactor (connection of the gas supply tubes through the top of the pouch). Said attachment means 50 consists of a male part 51 and of a female part 52 connected by a metal joint 53. The female part 52 made of plastic is welded to the pouch 40. The plastic material can advantageously be selected among the group consisting of polyethylene, polypropylene, the polyamides, polyether etherketone (PEEK), the copolymer of ethylene and polyvinyl alcohol (EVOH). The male part 51 is provided with two holes 54 wherein the gas supply tubes 30 are placed in a substantially fixed manner (the height of the tubes can however be modified under the influence of a physical force), and is placed on the female part 52. The metal connecting joint 53, consisting of two semicircles able to pivot about a common axis 55, and of a screw closure 56, allows the female and male parts to be united into a single block, the block being combined with the flexible pouch 40. The gas supply tubes can sometimes be placed within reinforcing bars to make them stiffer and stronger. Although FIGS. 3 and 4 do not show it, the gas supply tubes are held to a fixed base by means of a clamp-nut system.

FIGS. 5 and 6 illustrate the attachment means 60 according to the second currently preferred embodiment of the disposable bioreactor (connection of the gas supply tubes through the side of the pouch). Said attachment means 60 consists of a dual tube connector. This connector has the form of two ganged sprinkler hose connectors, that is it consists essentially of two male parts 61 of tubular shape each having a threaded end 61f. These male parts also include, perpendicular to their tube 61, a substantially flat surface 62 made of plastic, allowing the dual connector to be welded to the pouch 40. The plastic material of the substantially flat surface 62 can be advantageously selected from among the group consisting of polyethylene, polypropylene, the polyamides, polyether etherketone (PEEK), the copolymer of ethylene and polyvinyl alcohol (EVOH). The threaded shape of the ends 61f makes it possible to make a fluid-tight connection to the gas supply tubes. Although FIGS. 5 and 6 do not show them, reinforcing bars are connected to the non-threaded ends and are held to a fixed base by means of a clamp-nut system.

Thus, the invention makes it possible to obtain a disposable bioreactor of simple design, as well as a stirring system which has the advantage of being adaptable to any form of disposable bioreactor.

1. A disposable bioreactor including a flexible pouch made of a polymer material, wherein it also includes a single-use stirring system placed entirely within said pouch; said stirring system comprising a drive means and at least one stirring means driven in rotation by said drive means via magnetic coupling.

2. A bioreactor according to claim 1, wherein the stirring system also comprises a means of actuating the drive means consisting of a fluid or electricity (electrical charges) supply line.

3. A bioreactor according to claim 2, wherein the fluid is a gas selected from the group consisting of air, nitrogen, carbon dioxide, argon, diatomic oxygen and any mixture thereof.

4. A bioreactor according to claim 1, wherein said at least one stirring means comprises a rotation axle, said rotation axle having at least one metal surface capable of being attracted by a magnetized surface, and/or said rotation axle having at least one magnetized surface.

5. A bioreactor according to claim 1, wherein said at least one stirring means comprises at least one propeller having a hub and at least one blade.

6. A bioreactor according to claim 4, wherein said drive means is a turbine which includes vanes forming an integral part of a hollow shaft, said rotation axle of said at least one stirring means being placed within said hollow of the shaft and the inner wall of said hollow shaft having:
at least one metal surface attracted by said magnetized surface of the rotation axle of said at least one stirring means; and/or
at least one magnetized surface, attracting said at least one metal or magnetized surface of the rotation axle of said at least one stirring means.

7. A bioreactor according to claim 6, wherein the turbine also comprises a fixed body having substantially the shape of a closed cylinder, said fixed body forming an enclosure wherein the vanes are free to move.

8. A bioreactor according to claim 6, wherein at least one of said vanes is provided with at least one magnet cooperating with at least one counter-magnet positioned on the lower surface of said at least one stirring means to prevent friction between the drive means and said at least one stirring means, said lower surface corresponding to the surface of said at least one stirring means opposite the surface from which said at least one vane of said at least one stirring means projects.

9. A bioreactor according to claim 2, wherein said fluid supply line also serves as an aerator of the medium to be mixed within the pouch.

10. A bioreactor according to claim 1, wherein the height of said single-use stirring system in the pouch is variable and can be varied during stirring.

11. A single-use stirring system comprising:
a drive means, and
at least one stirring means driven in rotation by said drive means via magnetic coupling,
wherein said at least one stirring means includes a rotation axle, said rotation axle having at least one metal surface capable of being attracted by a magnetized surface, and/or said rotation axle having at least one magnetized surface (22); and in that said drive means is a turbine which includes vanes forming an integral part of a hollow shaft, said rotation axle of said at least one stirring means being placed within said hollow of the shaft and the inner wall of said shaft having:
at least one metal surface attracted by said magnetized surface of the rotation axle of said at least one stirring means; and/or
at least one magnetized surface, attracting said at least one metal or magnetized surface of the rotation axle of said at least one stirring means.

12. A stirring system according to claim 11, wherein said at least one stirring means includes at least one propeller having a hub and at least one blade.

13. A stirring system according to claim 11, wherein the turbine also comprises a fixed body having substantially the shape of a closed cylinder, said fixed body forming an enclosure wherein the vanes are free to move.

14. A stirring system according to claim 12, wherein at least one of said vanes is provided with at least one magnet cooperating with at least one counter-magnet positioned on the lower surface of said at least one stirring means to prevent friction between the drive means and said at least one stirring means, said lower surface corresponding to the surface of said at least one stirring means opposite the surface (24s) from which said at least one vane of said at least one stirring means projects.

15. A stirring system according to claim 11, wherein stirring system also comprises a means of actuating the drive means consisting of a fluid or electricity (electrical charges) supply line.

16. A bioreactor according to claim 2, wherein said at least one stirring means comprises a rotation axle, said rotation axle having at least one metal surface capable of being attracted by a magnetized surface, and/or said rotation axle having at least one magnetized surface.

17. A bioreactor according to claim 3, wherein said at least one stirring means comprises a rotation axle, said rotation axle having at least one metal surface capable of being attracted by a magnetized surface, and/or said rotation axle having at least one magnetized surface.

18. A bioreactor according to claim 2, wherein said at least one stirring means comprises at least one propeller having a hub and at least one blade.

19. A bioreactor according to claim 3, wherein said at least one stirring means comprises at least one propeller having a hub and at least one blade.

20. A bioreactor according to claim 4, characterized in that said at least one stirring means comprises at least one propeller having a hub and at least one blade.

21. A bioreactor according to claim 5, wherein said drive means is a turbine which includes vanes forming an integral part of a hollow shaft, said rotation axle of said at least one stirring means being placed within said hollow of the shaft and the inner wall of said hollow shaft having:
at least one metal surface attracted by said magnetized surface of the rotation axle of said at least one stirring means; and/or
at least one magnetized surface, attracting said at least one metal or magnetized surface of the rotation axle of said at least one stirring means.

22. A bioreactor according to claim 7, wherein at least one of said vanes is provided with at least one magnet cooperating with at least one counter-magnet positioned on the lower surface of said at least one stirring means to prevent friction between the drive means and said at least one stirring means, said lower surface corresponding to the surface of said at least one stirring means opposite the surface from which said at least one vane of said at least one stirring means projects.

23. A stirring system according to claim 12, wherein the turbine also comprises a fixed body having substantially the shape of a closed cylinder, said fixed body forming an enclosure wherein the vanes are free to move.

24. A stirring system according to claim 13, wherein at least one of said vanes is provided with at least one magnet cooperating with at least one counter-magnet positioned on the lower surface of said at least one stirring means to prevent friction between the drive means and said at least one stirring means, said lower surface corresponding to the surface of said at least one stirring means opposite the surface from which said at least one vane of said at least one stirring means projects.

25. A stirring system according to claim 12, wherein the stirring system also comprises a means of actuating the drive means consisting of a fluid or electricity (electrical charges) supply line.

26. A stirring system according to claim 13, wherein the stirring system (1) also comprises a means of actuating the drive means consisting of a fluid or electricity (electrical charges) supply line.

27. A stirring system according to claim 14, wherein the stirring system also comprises a means of actuating the drive means consisting of a fluid or electricity (electrical charges) supply line.
